(19) United States
(12) Patent Application Publication
(10) Pub. No.: US 2014/0296741 A1
(43) Pub. Date: Oct. 2, 2014

(54) METHOD AND APPARATUS FOR SUBSURFACE TISSUE SAMPLING

(71) Applicant: William G. Austen, Weston, MA (US)

(72) Inventor: William G. Austen, Weston, MA (US)

(73) Assignee: THE GENERAL HOSPITAL CORPORATION, Boston, MA (US)

(21) Appl. No.: 14/234,006

(22) PCT Filed: Jul. 20, 2012
(86) PCT No.: PCT/US12/47716

§ 371 Date: May 23, 2014

Related U.S. Application Data
(60) Provisional application No. 61/510,260, filed on Jul. 21, 2011.

(51) Int. Cl. A61B 10/02

(52) U.S. Cl. CPC A61B 10/0275; A61B 2010/0208; A61B 2010/0225
USPC 600/567

(57) ABSTRACT

Exemplary embodiments of method and apparatus are provided for removing a subsurface sample of a biological tissue while leaving the overlying tissue substantially undamaged. A hollow needle can be provided that includes an opening in the wall and a protruding cutting edge adjacent to the opening. A sliding sleeve can be provided over a portion of the needle. The needle and sleeve can be advanced into the tissue such that the cutting edge is deeper than the distal end of the sleeve. The needle may then be withdrawn while holding the sleeve stationary, such that a sample is severed by the cutting edge and directed into the hollow needle through the opening, until the cutting edge reaches the end of the sleeve. The sleeve and needle containing the sample can then be withdrawn from the tissue together.

### CROSS REFERENCE TO RELATED APPLICATION

The present application relates to and claims priority from U.S. Provisional Patent Application Ser. No. 61/510,260 filed Jul. 21, 2011, the disclosure of which is incorporated herein by reference in its entirety.

### FIELD OF THE DISCLOSURE

The present disclosure relates to methods and apparatus for removing or harvesting subsurface tissue or portions thereof, while reducing or avoiding significant damage to the overlying tissue.

### BACKGROUND INFORMATION

Procedures and devices for removing tissue samples, e.g. for cosmetic or diagnostic reasons, are commonly used in dermatology and other practice areas. For example, removal of tissue can be performed for cosmetic reasons, e.g., removal of fat to alter the appearance of a patient. Tissue samples can also be removed for diagnostic purposes, e.g., when performing biopsies to analyze and characterize tissue samples. Conventional tissue-removal procedures and devices can be disruptive to surrounding tissue and often includes many risks such as excessive bleeding, etc. For example, cosmetic procedures or biopsies can use an incision or remove portions of surface tissue, which can lead to scarring, infection, etc.

In certain applications, it may be desirable to obtain samples of skin or other tissues from particular depths below a tissue surface, such as dermal tissue located below the upper (epidermal) layer of skin or samples from certain tissue layers of other organs. Such samples may be used for analysis and diagnosis of certain conditions, or the tissue may be used for cultivation of larger tissue mass, e.g., to be used as graft material or the like. Removal of such subsurface tissue while avoiding removal of tissue at the surface can facilitate healing and/or improve appearance of the tissue in the sampled region.

Accordingly, there is a need for a simpler and safer method and apparatus for removal of subsurface tissue that addresses the issues and/or limitations described above.

### SUMMARY OF EXEMPLARY EMBODIMENTS

Exemplary embodiments of the present disclosure provide simple, inexpensive, and safe methods and devices for a removal of tissue samples from beneath the surface of skin, organs, or the like, while reducing or avoiding significant damage to the overlying tissue, such as the dermis and epidermis in skin.

An exemplary apparatus can be provided that includes a hollow needle and at least one cutting edge adjacent to an opening provided on a wall of the hollow needle. A sleeve can be provided around a portion of the needle, such that the sleeve is slidably translatable along the longitudinal axis of the needle. The distance from the central needle axis to the outer edge of the sleeve can be at least as large as a distance from the needle axis to the outer portion of the cutting edge, such that the perimeter of the cutting edge is covered or shielded by the sleeve when a distal end of the sleeve is positioned adjacent to the cutting edge.

The hollow needle can be configured to be inserted into a biological tissue such as skin, such that the needle penetrates the upper tissue layers. The size and geometry of the needle and cutting edge can be configured such that a portion of subsurface tissue can be cut from the surrounding tissue and enter the hollow core of the needle when the needle is withdrawn from the tissue through the surrounding sleeve. The sleeve can be configured to be partially inserted within the tissue along with the hollow needle. The sleeve can be held substantially stationary relative to the tissue as the needle is withdrawn, such that the cutting edge approaches and is covered or shielded by the distal end of the sleeve and no longer cuts off a portion of tissue when the needle is partially withdrawn from the tissue. The needle and sleeve can then be withdrawn completely from the tissue together. In this manner, a portion of tissue can be cut and removed from the surrounding tissue below the surface, while the sleeve can prevent or avoid removal of any tissue at and/or near the tissue surface by shielding or blocking the cutting edge as it approaches the tissue surface during withdrawal of the needle.

Adjustable arrangements can be provided to facilitate accurate insertion depths of the sleeve in the tissue and/or to limit the range of travel of the needle within the sleeve. Accordingly, embodiments of the present disclosure can facilitate removal of subsurface tissue portions or samples from a predetermined range of depths in the tissue.

In a further exemplary embodiment, a plurality of such hollow needles that include cutting edges can be affixed to a substrate. A plurality of corresponding sleeves can be provided around the needles. The substrate and needles can be arranged to control and/or limit the depth of penetration of the needles into the tissue when the substrate is placed on the tissue surface. The insertion depth of the sleeves can also be controlled by an appropriate arrangement/device. In this manner, a plurality of subsurface tissue portions can be removed or harvested simultaneously over one or more predetermined depth ranges. In certain embodiments, the size and shape of the opening, cutting edge, needle and/or sleeve can be configured to facilitate removal of particular subsurface tissue structures such as, e.g., bulbs of hair follicles or sweat glands.

In a still further exemplary embodiment, the apparatus can include a reciprocating arrangement affixed to the one or more needles. The reciprocating arrangement can include a motor or other actuator configured to repeatedly advance and withdraw the needles relative to the tissue. The reciprocating arrangement can also be configured to control the position of a sleeve positioned around each needle relative to the needle. The reciprocating arrangement can be provided in a housing that facilitates manipulation of the apparatus, e.g., placement of the apparatus on the tissue being treated and/or traversing the apparatus over the tissue. The housing can optionally be configured to stretch or otherwise stabilize the tissue proximal to the needle(s) being inserted, to reduce deformation of the tissue and/or improve accuracy of the placement of the needle(s) in the tissue. The reciprocating arrangement can further include a translational controller configured to translate the needles over the tissue in at least one direction, and optionally in two orthogonal directions, to facilitate removal or harvesting of subsurface tissue portions from larger regions of a donor tissue site without translating the entire apparatus over the tissue surface.

In yet further exemplary embodiments of the present disclosure, methods and apparatus can be provided to facilitate removal of particular biological structures from a tissue such as, e.g., hair bulbs or sweat glands from skin.

These and other objects, features and advantages of the present invention will become apparent upon reading the following detailed description of embodiments of the disclosure, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments, results and/or features of the exemplary embodiments of the present invention, in which:

FIG. 1 is a cross-sectional side view of an exemplary apparatus for removal of subsurface tissue in accordance with exemplary embodiments of the present disclosure;

FIGS. 2A-2D are schematic side views of the exemplary apparatus shown in FIG. 1 illustrating various steps for removal of subsurface tissue in accordance with exemplary embodiments of the present disclosure;

FIG. 3 is a cross-sectional side view of a second exemplary apparatus for removal of subsurface tissue in accordance with further exemplary embodiments of the present disclosure; and

FIG. 4 is a cross-sectional side view of a third exemplary apparatus for removal of subsurface tissue in accordance with still further exemplary embodiments of the present disclosure.

While the present disclosure will now be described in detail with reference to the figures, it is done so in connection with the illustrative embodiments and is not limited by the particular embodiments illustrated in the figures. It is intended that changes and modifications can be made to the described embodiments without departing from the true scope and spirit of the present disclosure as defined by the appended claims.

### DETAILED DESCRIPTION

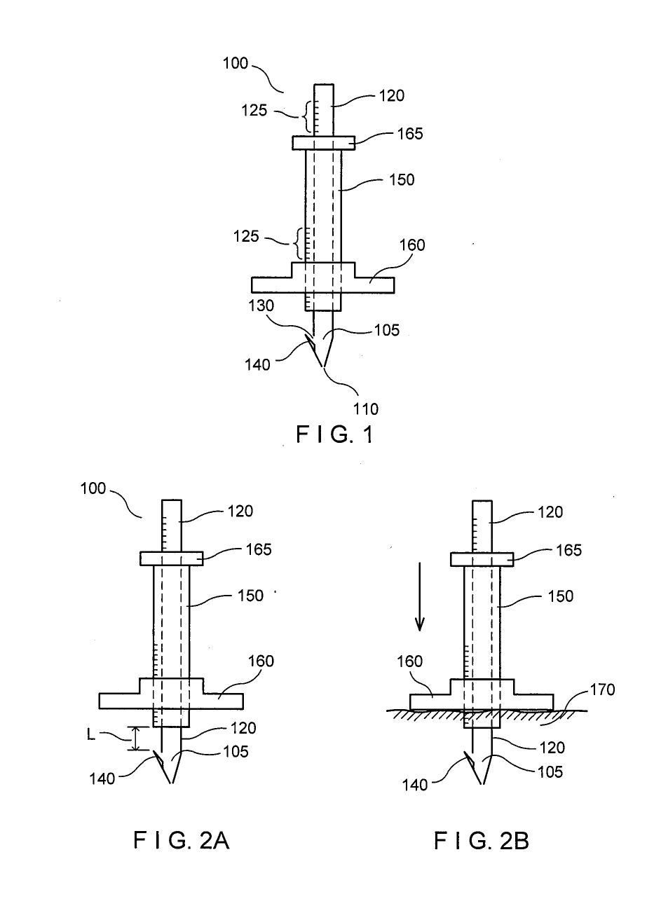

According to certain exemplary embodiments of the present disclosure, method and apparatus can be provided for reliably removing small tissue samples from a particular depth or depth range within a tissue structure. A side view of an exemplary apparatus 100 for removing a tissue sample is shown in FIG. 1 A. The exemplary apparatus 100 can include a hollow needle 120, having a central lumen or reservoir 105, which is configured to be inserted into a region of tissue to be analyzed. The diameter or width of the needle 120 can be, for example, about 1 mm in diameter. Larger or smaller diameters or widths may also be used in further embodiments. For example, needle widths that are significantly larger than about 1 mm, e.g., about 2-3 mm or more, may be capable of removing larger subsurface tissue portions. However, such wider needles may cause significant discomfort when inserted into tissue and/or may lead to slow healing and/or some scarring when removed from the tissue. Similarly, narrower needles may be better tolerated but may also be capable of removing only smaller tissue portions.

The distal end 110 of the needle 120 can be sharpened and/or otherwise configured to facilitate penetration of the needle 120 into a tissue site of interest. For example, the distal end 110 of the needle 120 may be pointed or tapered, as shown in FIG. 1. There may be an opening provided at the distal end 110 of the needle 120 in communication with the central lumen 105. In further embodiments, the distal end 110 of the needle 120 may be a solid point.

One or more openings 130 with an adjacent cutting edge 140, e.g., a flange or the like, can be provided in the wall of the needle 120. The upper portion of the cutting edge 140 can protrude outward from the outside of the needle 120, as shown in FIG. 1. For example, a cutting edge 140 with open sides can be formed, e.g., by cutting one or two slits through the needle wall just below the opening 130 to form a tab, and bending the tab outward from the center of the needle 120. The cutting edge 140 can also be formed by deforming the portion of the needle wall just below the opening 130 outwardly, to form a cutting edge 140 having closed sides, e.g., in a configuration similar to a single cutting edge on a conventional cheese grater.

Alternatively, the cutting edge 140 can be formed separately and attached to the needle 120 adjacent to the opening 130. Other configurations of the cutting edge 140 and adjacent opening 130 can also be used in accordance with embodiments of the present disclosure. For example, the upper border of the cutting edge 140 can be rounded, or angled, or it may have another shape. The

The exemplary apparatus 100 can also include a sleeve 150 that can be provided around at least a portion of the needle 120. For example, the sleeve 150 can have a shape of a hollow cylinder or tube, with an inside diameter or width that is substantially the same as an outside diameter or width of the needle 120. The sleeve 150 can be slidably translatable over the needle 120, e.g., it may be movable in a direction along a common longitudinal axis of the needle 120 and sleeve 150.

The outer wall of the needle 120 can have a substantially circular cross-sectional shape (e.g., it may be substantially cylindrical in shape), or other cross-section shapes may be used (e.g., square, rectangular, triangular, etc.). For example, a square cross-section can be provided, with an opening 130 and adjacent cutting edge 140 provided on two opposite sides of the needle 120, to facilitate harvesting or removal of more tissue with a single insertion and withdrawal procedure as described below. In further embodiments, the needle 120 can be provided with a square or triangular cross-sectional shape, and an opening 130 and adjacent cutting edge 140 may be provided on each of the 3 or 4 sides of the needle 120. The cross-sectional shape of the inner wall of the sleeve 150 (e.g., the walls defining a central lumen of the sleeve 150) can substantially correspond to the cross-sectional shape of the outer wall of the needle 120, e.g., such that the needle 120 substantially fills the sleeve 150 when inserted therethrough. Such corresponding shapes can facilitate relative motion of the needle 120 and sleeve 150 along a common longitudinal axis. Further, non-circular cross-sectional shapes of the sleeve 150 and needle 120 can be provided to inhibit rotation of the needle 120 around its longitudinal axis when it is inserted into the sleeve 150.

A wall thickness of the sleeve 150 can be substantially the same as, or slightly larger than, a distance that the upper portion of the cutting edge 140 protrudes outward from the outside surface of the needle 120. The proximal end of the needle 120 can protrude from the proximal end of the surrounding sleeve 150, as shown in FIG. 1. This configuration can facilitate independent manipulation and/or positioning of both the needle 120 and the sleeve 150. For example, the proximal end of the needle 120 and/or outer portions of the sleeve 150 can optionally include one or more markings 125 to indicate the position of the sleeve 150 relative to the needle 120. In this manner, the position of the sleeve 150 relative to the needle 120 can be ascertained when the distal end 110 of the needle 120 is inserted into a tissue, and a distal portion of the sleeve 150 may also be inserted into the tissue. The insertion depth of the needle 120 and/or sleeve 150 can also be determined by observing the position of the optional markings 125 on the needle 120 and/or sleeve 150 relative to the tissue surface and/or each other. Such markings 125 can be used with any of the embodiments described herein.

In further exemplary embodiments, a stop arrangement 160 can be provided on or proximal to a distal portion of the sleeve 150, e.g., as also shown in FIG. 1A. The stop arrangement 160 can be movably engaged with the outer surface of the sleeve 150, e.g., via frictional contact. In further exemplary embodiments, the stop arrangement 160 can be adjustably coupled to the sleeve 150 using a threaded screw arrangement, e.g., with screw threads on the outer surface of the sleeve 150 and corresponding threads provided on the inner surface of the stop arrangement 160. Rotating the stop arrangement 160 relative to the sleeve 150 can adjust the position of the stop arrangement 160 relative to the distal end of the sleeve 150. The markings 125 can be used to facilitate precise positioning of the stop arrangement 160. A locking arrangement (not shown) such as, e.g., a threaded nut or a clamp, can be provided on the sleeve 150 and/or stop arrangement 160 to prevent unwanted motion of the stop arrangement 160 relative to the sleeve 150 after it has been positioned. In certain embodiments, the stop arrangement 160 can be provided as a housing enclosing at least a portion of the sleeve 150, or optionally coupled or attached to such a housing. Such a stop arrangement 160 can be used with any of the embodiments described herein to limit or control the penetration depth of the sleeve 150.

In further exemplary embodiments, a collar arrangement 165 can be movably engaged or coupled to a proximal portion of the needle 120, e.g., as shown in FIG. 1A. The collar arrangement 165 can be movably engaged with and/or adjustably coupled to the outer surface of the needle 120, e.g., via frictional contact or a threaded screw arrangement. The markings 125 can be used to facilitate precise positioning of the collar arrangement 165. A locking arrangement (not shown) such as, e.g., a threaded nut or a clamp, can be provided on the needle 120 to prevent unwanted motion of the collar arrangement 165 relative to the needle 120 after it has been positioned. In certain exemplary embodiments, the collar arrangement 165 can extend beyond the proximal end of the needle 120, or be coupled to an extended protrusion, to provide a button or knob that can facilitate controlled motion or positioning of the needle 120. The collar arrangement 165 can be used with any of the embodiments described herein, e.g., to control the movement range of the needle 120 relative to the sleeve as described herein.

For example, the collar arrangement 165 can be adjusted to determine, constrain and/or limit the range of motion of the needle 120 relative to the sleeve 150. For example, the maximum distance that the tip 110 of the needle 120 protrudes past the distal end of the sleeve 150 can be limited by contact between the collar arrangement 165 and the proximal end of the sleeve 150. Similarly, the minimum distance between the tip 110 of the needle 120 and the distal end of the sleeve 150 can be achieved when the needle 120 is withdrawn from the proximal end of the sleeve 150 until the cutting edge 140 contacts the distal end of the sleeve 150.

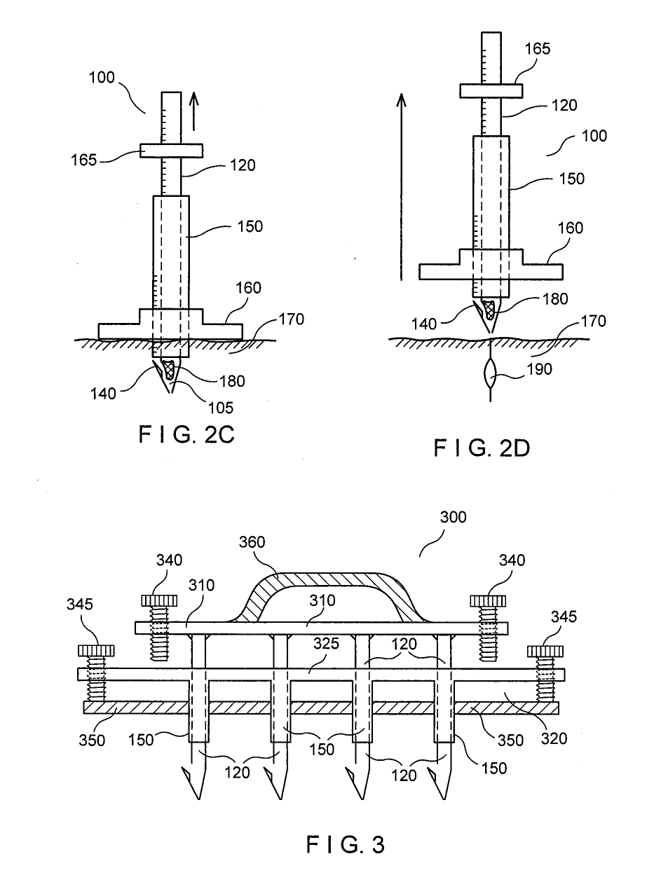

The exemplary apparatus 100 can be used to obtain subsurface tissue samples by first positioning the sleeve 150 relative to the needle 120 such that the distance between the upper portion of the cutting edge 140 and the distal end of the sleeve 150 substantially corresponds to a length L of the tissue sample to be collected, as shown in FIG. 2A. This distance can be set or adjusted, e.g., by appropriate positioning of the optional collar arrangement 165 on the needle 120. The exemplary apparatus 100 (e.g., the distal portions of the needle 120 and sleeve 150) can then be advanced into the tissue 170 until the upper portion of the cutting edge 140 is located at a depth substantially corresponding to the deepest portion of the tissue 170 to be sampled, as shown in FIG. 2B. This penetration depth can be set or adjusted, e.g., by appropriate positioning of the optional stop arrangement 160 on the sleeve 150. The position of the sleeve 150 relative to that of the needle 120 can be maintained as the apparatus 100 is inserted into the tissue 170, e.g., by pushing down on the proximal end of the needle 120, such that the collar arrangement 165 can simultaneously push on the proximal end of the sleeve 150.

The needle 120 can then be pulled back up through the tissue 170 while the sleeve 150 is held substantially stationary relative to the tissue 170, as shown in FIG. 2C. As the needle 170 is withdrawn, the cutting edge 140 can slice off or sever a tissue sample 180 from the tissue 170 proximal to the exterior of the needle 120. This tissue sample 180 can be directed into the hollow distal portion 105 of the needle 120 by the cutting edge 140, as shown in FIG. 2C. The needle 120 can be withdrawn until the cutting edge 140 is proximal or adjacent to the distal end of the sleeve 150, as shown in FIG. 2C.

Once the cutting edge 140 approaches or contacts the distal end of the sleeve, as shown in FIG. 2C, the cutting edge 140 can completely sever the tissue sample 180 from the surrounding tissue 170. The distal end of the sleeve 150 can also abut or cover the upper portion of the cutting edge 140. The apparatus 100 can then be removed from the tissue 170 by withdrawing the needle 120 and sleeve 150 together. The cutting edge 140 can facilitate withdrawal of the sleeve 150 by forcing the distal end of the sleeve 150 upward as the needle 120 is withdrawn from the tissue 170, as shown in FIG. 2D. The thickness of the sleeve 150 can cover the cutting edge 140 and prevent severing of further material from the tissue 170 as the needle 120 is fully withdrawn from the tissue 170. The tissue sample 180 that was severed from within the tissue 170 can be retained within the hollow needle 120, and subsequently removed for analysis or processing.

In further exemplary embodiments of the present disclosure, the apparatus 100 can be used to harvest a tissue sample 180 by first advancing the sleeve 150 towards the distal end of the needle 120 (or, equivalently, withdrawing the needle 120 from the proximal end of the sleeve 150) such that the distal end of the sleeve 150 is proximal to or contacting the upper portion of the cutting edge 140, e.g., as shown in FIG. 2D (without the tissue sample 180 present). The apparatus 100 can then be inserted into the tissue 170 to a desired depth, for example, to a depth where the distal end of the sleeve 150 is at or near the upper location of the tissue 170 to be sampled, e.g., in a position similar to that shown in FIG. 2C. The optional stop arrangement 160 can facilitate adjustment and/or control of this penetration depth by inhibiting further penetration when the lower surface of the stop arrangement 160 contacts the upper surface of the tissue 170.

The needle 120 can then be advanced further into the tissue 170, e.g., until the upper portion of the cutting edge 140 reaches a desired depth, e.g., a depth corresponding to a lower location of the region of tissue to be harvested, as shown, e.g., in FIG. 2B. Control or adjustment of this depth can be facilitated, e.g., by appropriate positioning of the collar arrangement 165 on the proximal portion of the needle 120. The needle 120 can then be retracted back up through the tissue 170 while the sleeve 150 is held substantially stationary relative to the tissue 170, as shown in FIG. 2C. As the needle 120 is withdrawn, the cutting edge 140 can slice off or separate a tissue sample 180 from the surrounding tissue 170 proximal to the exterior of the needle 120. This tissue sample 180 can be directed into the hollow distal portion 105 of the needle 120, as shown in FIG. 2C. The needle 120 can be withdrawn until the cutting edge 140 is proximal or adjacent to the distal end of the sleeve 150, as shown in FIG. 2C.

Once the cutting edge 140 and distal end of the sleeve meet, as shown in FIG. 2C, the cutting edge 140 can completely sever the tissue sample 180 from the surrounding tissue 170 and the apparatus 100 can then be removed from the tissue 170 with the tissue sample 180 retained within the hollow needle 120, as described above.

In further exemplary embodiments, a plurality of tissue samples 180 can be removed following a single insertion of the apparatus 100 into the tissue 170. For example, the needle 120 can be advanced into the tissue 170 as shown in FIG. C, and then withdrawn as shown in FIG. 2C while holding the sleeve 150 substantially stationary with respect to the tissue 170. These steps can be repeated, optionally rotating the needle 120 within the sleeve 150 in-between the needle withdrawal and advancement steps so that tissue samples 180 may be removed from different locations around the distal end of the sleeve 150.

The tissue 170 is shown in FIG. 2D after the tissue sample 180 has been severed and removed as described herein. The dark region in FIG. 2D represents a void 190 formed by removal of the tissue sample 180 from below the surface of the tissue 170 at a particular range of depths, as described herein. This void 190 may close partially or completely after the tissue sample 180 has been removed, depending on characteristics of the tissue 170. The line above and below the void 190 in FIG. 2D indicates where the distal end 110 of the needle 120 separated the tissue 170 upon insertion of the apparatus 100. This tissue can rejoin after the apparatus 100 is removed from the tissue 170, and the void 190 may also shrink and/or close fully after the sample 180 is removed. As further shown in FIG. 2D, the tissue sample 180 can be removed without removing material from the surface of the tissue 170. Accordingly, the region of tissue 170 may heal faster and/or be less prone to scarring or infection, even after a subsurface tissue sample 180 has been removed.

The exemplary method and apparatus described herein can allow a small tissue sample 180 to be removed from within a target tissue 170 at a predetermined or known range of depths, without removing any substantial amount of tissue 170 from the surface. The exemplary embodiments of the present disclosure thereby facilitates biopsy samples or other tissue portions 180 to be obtained and analyzed, where the tissue samples can be smaller than those used in conventional biopsy procedures and may be obtained from a particular depth within the tissue. The use of such depth-specific tissue samples 180 can facilitate healing and avoid the removal of excess tissue and/or formation of scars or markings resulting from such removal.

In a further exemplary embodiment, an exemplary tissue harvesting apparatus 300 (shown in FIG. 3) can be provided that includes a plurality of exemplary coring needle arrangements 100 as described herein. The needle 120 of each needle arrangement 100 can be affixed to a substrate 310, which may be a base, a plate, part of a housing, or the like. The exemplary needle arrangements 100 can be configured such that the sleeve 150 for each exemplary needle arrangement 100 can be operated simultaneously or in any controlled sequence.

For example, a sleeve arrangement 320 is shown in FIG. 3 that includes a plurality of sleeves 150 affixed or coupled to a sleeve base 325. The sleeve arrangement 320 can be slidably attached to the substrate 310 such that the (minimum) distance between the sleeve arrangement 320 and the needle substrate 310 can be controllably varied. For example, slidable or threaded adjusters 340 can be provided between the substrate 310 and the sleeve base 325, such that the advancement of the needles 120 through the sleeves 150 will be stopped or impeded when the distal end of the threaded adjusters 340 contact the upper portion of the sleeve arrangement 320 as shown in FIG. 3. For example, the threaded adjustors 340 coupled to the substrate 310 can limit the advancement distance of the needles 120 in the sleeves 150 in a manner similar to the operation of the collar arrangement shown in FIG. 2B and described above.

The exemplary apparatus 300 can optionally include a support base 350 as shown, e.g., in FIG. 3. The support base 350 can be configured as a plate or substrate that includes a plurality of openings therethrough corresponding to the locations of the sleeves 150 associated with the sleeve arrangement 320. The position of the support base 350 can be adjustable relative to sleeve arrangement 320 such that the protrusion distance of the distal ends of the sleeves 150 beyond the lower surface of the support base 350 can be varied. For example, threaded studs 345 can be provided that can be rotatably affixed to the upper surface of the support base 350 and which may pass through threaded openings in the sleeve base 325. In this exemplary configuration, the threaded studs 345 can be rotated to vary or adjust protrusion distance of the distal ends of the sleeves 150 beyond the lower surface of the support base 350, in a manner similar to that of the stop arrangement 160 illustrated in FIGS. 1 and 2 and described herein above. In further embodiments, the adjustable support base 350 and threaded studs 345 may not be present, and the sleeve arrangement 320 may be configured such that the distal ends of the sleeves 150 protrude to one or more desired fixed distances below the lower surface of the sleeve base 325. The lower surface of the sleeve base 325 and/or the support base 350, if present, can be substantially flat. In further embodiments, these lower surfaces may be contoured, e.g., convex or concave, to better conform to the particular surface shape of a region of tissue to be treated.

Each needle 120 of the exemplary needle arrangements 100 can be configured to pass through a sleeve 150 of the sleeve arrangement 320. The needle arrangements 100 in the apparatus 300 can be provided in a linear array, as shown in FIG. 3, in a 2-dimensional array, or in any desired pattern and/or spacing. A handle 360 or other protrusion can be affixed to the substrate 310 to facilitate manipulation of the substrate 310 and/or handling of an exemplary apparatus 300.

In an exemplary procedure, the minimum distance between the sleeve base 325 and the substrate 310 can be set to a predetermined distance, such that the distal end of each sleeve 150 is spaced apart from each cutting edge 140 by a maximum particular distance L, as shown in FIG. 2A. The spacing between the sleeve base 325 and the support base 350 can also be adjusted to control the penetration depth of the sleeves 150 into biological tissue. Accordingly, the apparatus 300 can then be inserted into the tissue to be harvested by pushing down on the substrate 310 until the lower surface of the support base 350 contacts the surface of the tissue, such that both the distal ends of the needles 120 and the distal portions of the sleeves 150 penetrate and enter the tissue to predetermined depths. This exemplary procedure is similar to the single-needle procedure illustrated in FIG. 2B. The substrate 310 can then be withdrawn from the tissue while holding the sleeve arrangement 320 substantially stationary with respect to the tissue, e.g., by pulling upward on the handle 360 and/or substrate 310, such that each needle 120 is withdrawn through a corresponding sleeve 150 of the sleeve arrangement 320 until the cutting edge 140 is proximal to the distal end of the sleeve 150, similar to the single-needle configuration shown in FIG. 2C. The entire apparatus 300 can then be withdrawn from the tissue, whereby each needle 120 will contain a piece of subsurface tissue 180 as shown, e.g., in FIG. 2D.

In one embodiment, the length of each sleeve 150 protruding from the lower surface of the sleeve base 325 can be configured to be approximately the same distance as the depth of the upper portion of the tissue samples to be harvested. In this manner, the sleeve arrangement 320 can be inserted into the tissue donor site such that the lower surface of the sleeve base 325 or support base 350, if present, contacts the tissue surface, and the distal end of each sleeve 150 will be at a desired depth within the tissue. The length of the needles 120 protruding from the substrate 310 can also be selected or adjusted such that the cutting edge 140 is at the desired distance L from the distal end of each sleeve 150 when the substrate 320 and sleeve plate 320 are as close to each other as possible (e.g., the distal ends of the needles 120 extend as far as possible through the distal ends of the sleeves 150). In use, the substrate 310 and sleeve arrangement 320 can be pressed onto the donor site until the sleeve base 325 is contacting the tissue surface and the needles 120 are fully inserted into the tissue. The substrate 310 can then be lifted away from the tissue surface while maintaining the sleeve base 325 against the surface. This allows the needles 120 to be retracted partially up into the sleeves 150 while the sleeves 150 remain substantially stationary with respect to the tissue. Once the cutting edges 140 reach the distal ends of the sleeves 150, e.g., as shown in FIG. 2C, the sleeve arrangement 320 can be withdrawn from the donor site together with the needles 120 to remove the plurality of tissue samples that were cut from surrounding tissue and retained in the needles 120 as described herein.

In further exemplary embodiments, one or more pressure or force sensors or the like can be provided in the apparatus 100 or 300. Such a sensor can include a piezoelectric material or wire, or the like, and may be used to assist in controlling the depth of the tissue harvesting. For example, a sensor may be provided to indicate when different tissue structures are be reached by the needle 120 and/or sleeve 150 during their insertion, e.g., based on a change in the amount of pressure or force needed to insert the needle arrangement 100 further into the tissue.

The exemplary apparatus 300 can include a plurality of needle arrangements 100 can be used to selectively harvest dermal tissue from a donor site. The use of a plurality of the exemplary needle arrangements 100 can facilitate faster harvesting of a larger amount of dermal tissue samples 180.

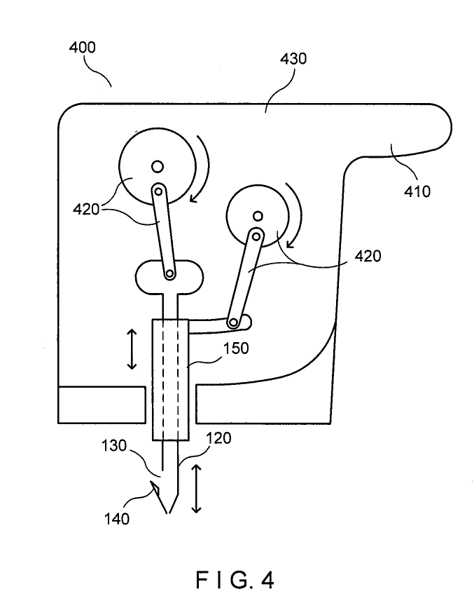

A still further exemplary apparatus 400 is shown in FIG. 4 that can include one or more needles 120, with each needle 120 provided with at least one cutting edge 140, and corresponding sleeve 150, e.g., as described herein and shown in FIGS. 1-3. The one or more needles 120 and corresponding sleeves 150 can be mechanically coupled to at least one reciprocating arrangement 420 provided within a housing 430. The housing 430 can also include a handle 410. The reciprocating arrangement 420 can be configured to displace the needle 120 back and forth along a direction that can be substantially parallel to the axis of the needle 120. For example, the reciprocating arrangement 420 can be powered by a motor or the like, and controlled by a switch that can turn the reciprocating arrangement 420 on and off, and may further control the reciprocating frequency.

The exemplary apparatus 400 can further include adjusting arrangements configured to adjust or control the maximum protrusion distance of the needle(s) 120 and/or the sleeve(s) 150 below the lower surface of the housing 430. The reciprocating arrangement 420 can be further configured to controllably translate the sleeve(s) 150 relative to the needle(s) 120. Accordingly, the reciprocating arrangement 420 can be configured to control the positions of both the needle 120 and the sleeve 150 to harvest subsurface tissue portions 180, e.g., as illustrated in FIGS. 2A-2D. Accordingly, the reciprocating arrangement 420 can be configured to control the range of depths over which the cutting edge 140 cuts a tissue portion 180 from the surrounding tissue 170, as shown in FIGS. 2A-2D.

The exemplary apparatus 400 can be traversed over a region of skin to be treated such that the one or more needles 120 and sleeve(s) 150 can be repeatedly inserted and withdrawn from the tissue, removing a portion 180 of subsurface tissue upon each withdrawal as described herein. The penetration depth of the needle(s) 120 and sleeve(s) 150 can be determined by the configuration of the reciprocating arrangement 420.

In a further exemplary embodiment, the reciprocating arrangement 420 can further include a translational mechanism configured to translate the one or more needles 120 over the surface of the tissue 170 in one or two orthogonal directions. For example, the reciprocating arrangement 420 can be configured to translate the one or more needles 120 over an area of the tissue 170 while the apparatus 400 is held stationary with respect to the tissue surface at a donor or treatment site 170. In one exemplary embodiment, the reciprocating arrangement 420 can be configured to translate the one or more needles 120 along a single direction to harvest subsurface tissue portions 180 along one or more rows. The apparatus 400 can optionally be translated over the tissue surface after such rows are formed, e.g., in a direction that is not parallel to the row, to remove or harvest tissue portions 180 from a larger area of the donor tissue site 170.

In further exemplary embodiments of the present disclosure, any of the exemplary apparatuses described herein can be configured to remove or harvest subsurface tissue portions 180 from a plurality of locations in any of a variety of spatial distributions, where each location can correspond to a single insertion and withdrawal of a single needle 120. For example, the tissue portions 180 can be removed or harvested from a plurality of locations configured as one or more rows, a regular two-dimensional pattern, a random distribution, or the like. Such patterns or spatial distributions of tissue harvesting or removal sites can be generated based on, e.g., the configuration of the one or more needles 120 provided, the properties of the reciprocating arrangement 420, and/or the rate of translation of the exemplary apparatus 400 over the tissue surface.

In still further exemplary embodiments, the housing 430 can be configured to stretch skin or other tissue 170 when the apparatus 400 is placed on the tissue 170 to be treated. Such stretching can facilitate mechanical stabilization of the tissue 170, e.g., to reduce or avoid deformation of the tissue 170 while the needles 120 are inserted into and withdrawn from the tissue 170. Such stretching of the tissue 170 can also reduce the effective size of the disrupted region of the upper tissue layers formed by the apparatus when the tissue 170 is allowed to relax after treatment, and it may provide more precise control of the depth from which the tissue samples 180 are obtained by reducing deformation of the pliable tissue during the harvesting procedure. Alternatively, the surface of the tissue 170 to be treated can be stretched or stabilized using other techniques prior to and/or during treatment of the region in accordance with any of the exemplary embodiments described herein.

The exemplary methods and apparatus described herein can be used to extract or harvest particular biological structures from tissue. Many biological structures of interest may be located at a particular depth or range of depths below the tissue surface. Embodiments of the present disclosure provide methods and apparatus that facilitate removal of such subsurface structures, or portions thereof, while leaving the overlying (e.g., surface-region) tissue substantially undisturbed or undamaged. This can be achieved by removing subsurface tissue samples, while avoiding or minimizing removal of tissue from the overlying surface region of tissue as described herein.

For example, exemplary embodiments of the present disclosure may be used to extract bulbs of hair follicles from skin tissue (e.g., on the scalp) for transplantation to other areas of the skin or scalp. In one embodiment, the exemplary apparatus 100, 300 can be adjusted to obtain tissue samples 180 from a range of depths between about 4 mm and 6 mm, corresponding to the approximate depth of hair bulbs below the skin surface. For this exemplary procedure, the size of the opening 130 can be configured to be larger than the size of a typical hair bulb, e.g., greater than about 0.3-0.5 mm.

Although each tissue sample 180 harvested from such depth may not contain a bulb, the procedure described herein can be performed many times over an area of skin rapidly and without significant scarring or disruption of the upper skin region. Accordingly, a number of hair bulbs can be extracted from skin tissue in a reasonable time interval using the exemplary methods and apparatus described herein. The tissue samples 180, which may contain hair bulbs, can then be removed from the apparatus 100, 300 so that the tissue sample 180 may optionally be transplanted to a different region of the scalp or skin. For example, the needle(s) 120 can be reinserted into a recipient site of skin to an appropriate depth, and a pressurized fluid can be applied to the proximal end(s) of the needle(s) 120 to eject the tissue sample(s) 180 into the skin. Alternatively, a low pressure or vacuum can be applied to the proximal end(s) of the needle(s) to draw the tissue sample(s) 180 out of the needle(s) 120. The tissue sample(s) 180 can then be implanted into the recipient site using a separate apparatus.

In a further exemplary embodiment, the exemplary methods and apparatus described herein can be used to remove sweat glands from skin tissue. For example, the depth range at which the tissue samples 180 are extracted can be configured to be at or proximal to the lower portion of the dermis where sweat gland may be located, and the size of the opening 130 can be configured to be similar in width or slightly larger than the size of a typical sweat gland. For example, the opening 130 in the apparatus 100, 300, 400 can be greater than about 0.2 mm wide, e.g., between about 0.4 mm and 0.8 mm wide, to facilitate removal of sweat glands within the tissue samples 180. Exemplary embodiments of the present disclosure can facilitate removal of sweat glands from skin tissue with negligible disruption of the upper skin region. Removal of a portion of the sweat glands can reduce the rate of perspiration of the target area, e.g., in the armpits.

The foregoing merely illustrates the principles of the present disclosure. Various modifications and alterations to the described embodiments will be apparent to those skilled in the art in view of the teachings herein. It will thus be appreciated that those skilled in the art will be able to devise numerous techniques which, although not explicitly described herein, embody the principles of the present disclosure and are thus within the spirit and scope of the present disclosure. All patents and publications cited herein are incorporated herein by reference in their entireties.

1. An apparatus to measure a flow of fluid within an anatomical structure, comprising:
at least one probe first arrangement which is structured to be insertable into a vessel and configured to direct at least one radiation to at least one portion of the anatomical structure;
at least one detector second arrangement which is configured to detect an interference between a first radiation provided from the fluid via the first arrangement and a second radiation provided from a reference as a function of at least one wavelength of at least one of the first radiation or the second radiation; and
at least one computer third arrangement which is configured to determine characteristic of the fluid as a function of a change in the interference obtained at at least two different points in time.

2. The apparatus according to claim 1, wherein the at least one third arrangement determines the at least one characteristic that includes the flow as a function of an intensity of the interference.

3. The apparatus according to claim 1, wherein the at least one characteristic comprises particular parameters which includes at least one of flow, viscosity, velocity, coronary flow reserve, fractional flow reserve, coronary flow velocity reserve, average peak velocity, maximum peak velocity, average, peak velocity or pressure of the fluid within the vessel.

4. The apparatus according to claim 3, wherein the at least one characteristic comprises a multi-dimensional distribution of the particular parameters.

5. The apparatus according to claim 1, wherein the at least one third arrangement determines the at least one characteristic at multiple longitudinal locations within the vessel.

6. The apparatus according to claim 5, wherein the at least one third arrangement determines (i) the at least one characteristic by making measurements, with the probe arrangement at locations that are proximal and distal to stenosis, blockage or stented segment, and (ii) a further characteristic as a function of the measurements.

7. The apparatus according to claim 1, wherein the at least one third arrangement is further configured to determine geometry of a wall of the vessel.

8. The apparatus according to claim 7, wherein the property of the wall is a luminal contour or a bio-mechanical property of the wall, or a tissue characteristic of the wall.

9. The apparatus according to claim 1, wherein the at least one third arrangement determines the at least one characteristic of a wall of the vessel.

10. The apparatus according to claim 1, wherein the at least one first arrangement includes at least one of a catheter, a wire or a sheath.

11. The apparatus according to claim 1, wherein the fluid comprise at least one of blood, transparent medium, or a combination thereof.

12. (canceled)

13. The apparatus according to claim 1, wherein a wavelength of at least one of the first radiation or the second radiation varies over time.

14. The apparatus according to claim 1, wherein the at least one second arrangement includes at least one array of detectors, each configured to detect a separate wavelength band of the interference.

15. The apparatus according to claim 1, wherein the at least one third arrangement determines the at least one characteristic using a correlation procedure.

16. The apparatus according to claim 1, wherein the at least one third arrangement determines the at least one characteristic is determined as a function of a distance of the fluid being measured from the at least one first arrangement.

17. The apparatus according to claim 1, wherein the at least one third arrangement is further configured to extrapolate further characteristics of the fluid where the fluid is not measured based on the at least one characteristic and information regarding a property of a wall of the vessel.

18. The apparatus according to claim 1, wherein the at least one characteristic is a pressure of the fluid within the vessel, and wherein the at least one third arrangement determines the pressure as function of a property of a wall of the vessel.

19. The apparatus according to claim 1, wherein the at least one third arrangement determines the at least one characteristic by analyzing a speckle pattern of an image associated with the fluid.

20. The apparatus according to claim 1, wherein the at least one third arrangement is further configured to determine at least one three-dimensional information of a wall of the vessel using the interference.

21. The apparatus according to claim 1, wherein the at least one first arrangement is configured to be immobile during operation of the apparatus.

22. (canceled)

23. The apparatus according to claim 1, wherein the at least one third arrangement determines the at least one characteristic synchronously with a further physiological measurement which is determined by at least one fourth arrangement.

24. The apparatus according to claim 23, wherein the further physiological measurement is at least one of EKG, heart rate, systolic or diastolic blood pressure, maximal flow, minimal flow, arterial pressure or a pressure measurement.

25. The apparatus according to claim 1, wherein the at least one third arrangement determines the at least one characteristic at least one of before or after an administration of a pharmacologic agent.

26. The apparatus according to claim 1, wherein the at least one first arrangement directs the at least one radiation to the at least one portion along an axis which is approximately perpendicular to the direction of extension of the at least one first arrangement.

27. The apparatus according to claim 1, wherein the at least one third arrangement generates an audible sound based on the at least one characteristic.

28. The apparatus according to claim 27, wherein the at least one first arrangement is positioned within the apparatus is based on the sound.

29. The apparatus according to claim 1, wherein the at least one third arrangement generates a fractional flow reserve based on a pressure within the vessel.

30. The apparatus according to claim 1 further comprising a further arrangement for measuring pressure.

31. The apparatus according to claim 30, wherein the further arrangement is configured to generate information regarding the pressure based on the at least one electromagnetic radiation transmitted through the at least one first arrangement which includes a catheter.

32. The apparatus according to claim 30, wherein the further arrangement comprises at least one of a Fabry-Perot or a fiber grating sensor.

33. A method to measure a flow of fluid within an anatomical structure, comprising:
detecting an interference between a first radiation provided from the fluid via at least one first probe arrangement and second a second radiation provided from a reference path as a function of wavelength thereof, wherein the at least one first probe is inserted into a vessel and configured to direct at least one radiation to at least one portion of the anatomical structure; and
determining at least one characteristic of the fluid using the interference.
